(19) United States
(12) Patent Application Publication
(10) Pub. No.: US 2021/0255106 A1
(43) Pub. Date: Aug. 19, 2021

(54) CELL PURIFICATION DEVICE

(71) Applicant: SHIMADZU CORPORATION, Kyoto-shi (JP)

(72) Inventors: Masayuki NISHIMURA, Columbia, MD (US); Akihiro ISHIKAWA, Kyoto-shi (JP)

(73) Assignee: SHIMADZU CORPORATION, Kyoto-shi (JP)

(21) Appl. No.: 17/168,465

(22) Filed: Feb. 5, 2021

Related U.S. Application Data
(60) Provisional application No. 62/977,983, filed on Feb. 18, 2020.

(51) Int. Cl. G01N 21/64; B01L 3/00; G01N 21/03

(52) U.S. Cl. CPC G01N 21/6486; B01L 3/502715; B01L 2300/168; G01N 21/6428; B01L 2300/0654; G01N 21/0303

(57) ABSTRACT

A device capable of targeting and selectively removing (killing) an unnecessary cell that remains in a cell population induced to differentiate from pluripotent stem cells and causes tumorigenesis after transplantation, and automatically and efficiently collecting an object cell. The device includes a holder configured to hold a cell suspension containing cells and an antibody-photosensitizer conjugate obtained by conjugating a targeting molecule that binds to a surface protein of a cell to be removed and a fluorescently labeled substance, a radiation light source configured to radiate radiation to change a physical property of the fluorescently labeled substance contained in the holder, a detector configured to detect abundance of the cells in the holder, and a control/processing unit configured to control the radiant light source.

### DETAILED DESCRIPTION OF THE INVENTION

### Technical Field

The present invention relates to a device configured to kill a specific cell among cells cultured on a cell culture vessel and to extract an object cell, and a device such as a light source device or a cell culture vessel used to conduct that, for example.

### BACKGROUND ART

In recent years, research and development of regenerative medicine technology and drug discovery using somatic stem cells, embryonic stem cells, and induced pluripotent stem cells have been actively carried out. In this type of research and development, it is extremely important to be able to efficiently mass-produce object cells and tissues.

When cell culture is performed, it is common to perform subculture by cutting out a portion of a cell aggregate (colony) proliferated in a culture medium, replanting it in a new culture medium, and culturing it again when the cell aggregate (colony) grown in the culture medium satisfies a predetermined seeding density or area value, for example.

At present, subculture of the proliferated cells relies exclusively on manual operation, but this requires time and effort, and may cause irregularities in the sizes of the cut-out cells, which may result in variations in the state of growth of the subcultured cells.

Cases have been reported in which undifferentiated cells remain in a differentiated cell population and cause tumorigenesis (teratoma, carcinogenesis) when pluripotent stem cells such as ES cells or iPS cells are cultured under conditions for differentiating them into cells such as myocardium or nerves (Non-Patent Document 1). Furthermore, iPS cells are artificially reprogrammed cells, and thus there are safety issues such as the risk of tumorigenesis due to introduction of proto-oncogenes such as c-Myc or use of viral vectors, and the risk of tumorigenesis due to resistance to differentiation depending on the type of deriving somatic cells to be the derivation.

Even in the elucidation of pathological conditions using disease-specific iPS cells derived from patients and the development of drug discovery for the disease, contamination of undifferentiated cells other than the object differentiated cells causes other cell types, and thus accurate experiments cannot be performed due to mixing of cells other than the object cells. Similarly, in the application of human ES cells and iPS cells in terms of supplying normal human cells to drug efficacy and toxicity tests in drug discovery, contamination of undifferentiated cells other than the target differentiated cells causes other cell types, and thus it is conceivable that mixing of cells other than the target cells reduces the accuracy of measurement and the reliability thereof.

Therefore, when cells that have failed in differentiation into specific tissues or organs or have not differentiated or unnecessary cells such as tumorigenic cells are detected, as a countermeasure, the culture vessel containing these unnecessary cells may be discarded. However, it leads to a decrease in the recovery rate of normally differentiated object cells or tissues, and increases the cost of regenerative medicine.

Thus, it is desirable to improve the recovery rate of object cells or tissues without wasting the remaining cells by killing or removing the unnecessary cells existing in the culture vessel.

As a method for selectively killing unnecessary cells in a culture vessel, a method for radiating active energy rays such as visible light, ultraviolet rays, infrared rays, or radiation has been proposed (Patent Document 1). That is, a photo-acid-generating agent that generates an acidic substance by being irradiated with active energy rays such as visible light, ultraviolet rays, infrared rays, or radiation is applied to a surface of the culture vessel in advance, and the active energy rays are radiated for about 10 seconds to 10 minutes to a portion in which cells to be killed among the cells cultured in this culture vessel exist such that the acidic substance is generated to kill the target cells. A digital micromirror device (DMD), a liquid crystal shutter array, an optical spatial modulation element, a photomask, or the like is used to control a region to be irradiated with the active energy rays.

The method disclosed in Patent Document 1 requires a long time to radiate the active energy rays in order to kill the target cells, and it can be said that there is still room for improvement for the upcoming mass production of regenerative medicine cells. In addition, in a micro-projection system using a DMD or the like, most of energy supplied from an active energy source (light source) is wasted. Moreover, it is difficult to keep the intensity distribution of the active energy rays radiated to the photo-acid-generating agent uniform.

It is also conceivable to directly irradiate the unnecessary cells with the active energy rays such as high-energy pulse lasers in order to speed up a process of killing the unnecessary cells. However, the active energy rays to be radiated need to hit the cell nuclei, and the cells cannot be surely killed unless the target cells are irradiated a plurality of times. Furthermore, there is an essential problem that the thermal effect on the object cells around the unnecessary cells that are directly irradiated with the active energy rays is unavoidable.

### PRIOR ART

### Patent Document

[Patent Document 1] International Publication No. 2011/125615

### Non-Patent Document

[Non-Patent Document 1] Miura, K. et al., Nat. Biotechnol. 27: 743-745 (2009)

### SUMMARY OF THE INVENTION

### Problem to be Solved by the Invention

The present invention aims to provide a device capable of targeting and selectively removing (killing) unnecessary cells remaining in a cell population induced to differentiate from specific cells and including cells that have failed to differentiate or have not differentiated into desired tissues or organs, tumorigenic cells, etc., and automatically and efficiently collecting an object cell.

### Means for Solving the Problem

The present invention aims to provide a device using photoimmunotherapy that enables targeted killing of a specific cell that has bound to an antibody-IR700 conjugate by activating IR700 by irradiation with near-infrared light, focusing on an antibody-photosensitizer conjugate (antibody-IR700 conjugate) obtained by conjugating an antibody or another targeting molecule that targets a cell surface protein with a chemical substance that is a water-soluble phthalocyanine derivative called IR700.

Therefore, the present invention is:

a purification device configured to purify cells, the purification device including:

a holder configured to hold a cell suspension containing cells and an antibody-photosensitizer conjugate obtained by conjugating a targeting molecule that binds to a surface protein of a cell to be removed and a fluorescently labeled substance;

a radiation light source configured to radiate radiation to change a physical property of the fluorescently labeled substance contained in the holder;

a detector configured to detect abundance of the cells in the holder; and

a control/processing unit configured to control the radiant light source; wherein

the antibody-photosensitizer conjugate kills the cell to be removed by a change of the physical property of the fluorescently labeled substance; and

the control/processing unit is configured to determine whether or not the cell to be removed has been killed based on the abundance of the cells detected by the detector, and to continue irradiation with the radiation from the radiation light source when the cell to be removed has not been killed;

the purification device wherein

a container containing the cell suspension containing the cells, a conjugate storage configured to store the antibody-photosensitizer conjugate, and the holder are connected by flow paths; and

the flow paths include pumps configured to introduce the cell suspension and the antibody-photosensitizer conjugate into the holder;

the purification device, wherein the control/processing unit is configured to radiate radiation having enough energy to excite the fluorescently labeled substance but not kill the cell to be removed before killing the cell to be removed by radiating radiation to change the physical property of the fluorescently labeled substance, and to detect a position of the cell to be removed in the holder and then irradiate the position with radiation having enough energy to kill the cell to be removed;

the purification device, wherein the control/processing unit is configured to determine whether or not the cell to be removed has died based on a presence of fluorescence detected by radiating radiation having enough energy to excite the fluorescently labeled substance but not kill the cell to be removed after radiating radiation having enough energy to kill the cell to be removed;

the purification device, wherein the holder includes a cell container containing the cell suspension containing the cells;

the purification device further including:

a liquid feeding pump configured to flow the cell suspension; wherein

the holder forms a flow path including an inlet for taking in the cell suspension, an outlet for discharging the cell suspension containing purified cells, and the liquid feeding pump; and

the control/processing unit is configured to drive the liquid feeding pump to discharge the cell suspension in the flow path from the outlet when determining that removal of the cell to be removed has been completed based on the abundance of the cells detected by the detector;

the purification device further including:

a cell incubator configured to culture cells; wherein

the flow path forms a circulation flow path;

the cell incubator is provided in a return flow path of the circulation flow path; and

the control/processing unit is configured to drive the liquid feeding pump to feed the cell suspension in the flow path to the cell incubator when determining that the cell to be removed has died based on detected fluorescence abundance;

the purification device including:

an analyzer configured to analyze the cell suspension containing the antibody-photosensitizer conjugate and a ligand of the antibody-photosensitizer conjugate;

a flow path configured to supply the cell suspension to the analyzer; and

a switching valve configured to selectively switch any flow path to the analyzer or the cell incubator; wherein

the switching valve is provided in the return flow path of the circulation flow path;

the purification device, wherein the analyzer includes a liquid chromatograph mass spectrometer;

the purification device, wherein the radiation radiated by the radiation light source has a wavelength of 660 to 740 nm;

the purification device, wherein

the radiation radiated by the radiation light source includes near-infrared light; and

the fluorescently labeled substance activated by irradiation with the near-infrared light includes a phthalocyanine dye;

the purification device, wherein the phthalocyanine dye is IRDye® 700; and

the purification device, wherein a ligand of the antibody-photosensitizer conjugate is a C14H33NO10S3Si fragment.

Note that the cells in the present invention include not only cultured cells but also isolated cells such as pancreatic islets.

The detector includes a fluorescence detector configured to detect fluorescence emitted from the fluorescently labeled substance, or a cell imager.

A conventionally known mechanism of photoimmunotherapy is as follows:

Photoimmunotherapy (PIT), which is a new cancer treatment method discovered by Senior Researcher Hisataka Kobayashi et al. of the National Cancer Institute, is a therapeutic method that uses, as a drug, an antibody-photosensitizer conjugate (antibody-IR700 conjugate) to which a chemical substance that is a water-soluble phthalocyanine derivative called IR700 binds, and has extremely few side effects because it is not toxic to anything other than cancer cells (for example, U.S. Patent Application Publication Nos. 2017/0122853, 2016/001589, 2014/0120199, 2013/0336995, 2012/0010558 (Kobayashi et al.), and Non-Patent Document “Kazuhide Sato et al., ACS Cent. Sci. 2018, 4, 1559-1569”). Antibodies that are used in PIT and target cancerous cells and light absorbers/photosensitizers of phthalocyanine dye such as IRDye® 700 form antibody-photosensitizer conjugates. When these antibody-photosensitizer conjugates are injected into a patient and pass through the bloodstream to reach a tumor containing cancer cells, they leak from permeable blood vessels near the tumor and then bind to antigens, cell surface proteins that are specifically present in cancer cells.

After the antibody-photosensitizer conjugates bind to the cancer cells, near-infrared light is radiated to the cancer cells from either inside or outside the body. The near-infrared light is highly permeable to living organisms, and thus it is transmitted without causing damage to living tissues and the like. Irradiation with this near-infrared light activates IRDye® 700 and induces an axial ligand (C14H33NO10S3Si fragment) release reaction such that the shapes of the antibodies and antigens physically change, and physical stresses within cell membranes that lead to an increase in transmembrane water are induced. When water enters the cancer cells, they swell, and the internal cell pressure rises such that the cancer cells rupture and die. When the cancer cells rupture, internal substances inside the cancer cells are released into extracellular spaces. At that time, a body's immune system detects the internal substances and cell debris as “foreign substances”, and thus an immune response that promotes cancer destruction is activated. Specifically, T-lymphocytes, which are a type of white blood cell, and thymocytes (“T cells”) attack and destroy the cell debris. FIG. 4 shows a mechanism of cancer cell death.

Therefore, the “cell to be removed” in the present invention refers to any cell that has become a tumor, and can include cancer cells, for example. Specific examples include squamous cell carcinoma, but are not limited to this.

A protein or peptide that is a targeting molecule that binds to the surface protein (marker molecule) of the cell to be removed varies depending on the type of marker molecule, and examples thereof include cetuximab that binds to an epidermal growth factor receptor (EGFR). However, the present invention is not limited to cetuximab, but any antibody that binds to a marker molecule that is more strongly expressed than normal cells may be used. For example, panitumumab, zalutumumab, nimotuzumab, tositumomab, rituximab, ibritumomab tiuxetan, daclizumab, gemtuzumab, alemtuzumab, a CEA-scan Fab fragment, OC125, ab75705, B72.3, bevacizumab, basiliximab, nivolumab, pembrolizumab, pidilizumab, MK-3475, BMS-936559, MPDL3280A, ipilimumab, tremelimumab, IMP321, BMS-986016, LAG525, urelumab, PF-05082566, TRX518, MK-4166, dacetuzumab, lucatumumab, SEA-CD40, CP-870, CP-893, MED16469, MEDI6383, MEDI4736, MOXR0916, AMP-224, PDR001, MSB0010718C, rHIgM12B7, ulocuplumab, BKT140, varlilumab, ARGX-110, MGA271, lirilumab, IPH2201, AGX-115, emactuzumab, CC-90002, MNRP1685A, or an antigen-binding fragment thereof, which is described in Japanese Translation of PCT International Application Publication Nos. 2014-523907 and 2018-528268, may be selected.

The protein or peptide of the present invention can be produced by a known (poly) peptide synthesis method. The peptide synthesis method may be either a solid-phase synthesis method or a liquid-phase synthesis method, for example. The target peptide can be produced by condensing a partial peptide that can constitute the peptide of the present invention or amino acid with a residual portion, and removing a protecting group when the product has the protecting group.

The peptide obtained in this manner can be purified and isolated by a known purification method. Examples of the purification method include solvent extraction, distillation, column chromatography, liquid chromatography, recrystallization, and combinations thereof.

When the peptide obtained by the above method is in a free form, the form can be converted to a suitable salt by a known method or a method analogous thereto. Conversely, when the peptide is obtained in the form of a salt, the salt can be converted to a free form or another salt by a known method or a method analogous thereto.

The mode of binding of the protein or peptide of the present invention with one or more components is not particularly limited. The binding may be direct or indirect via a linker etc. The binding may be by covalent binding, non-covalent binding, or a combination thereof. One or more components may be directly or indirectly bonded at an N-terminal, a C-terminal, or another position of the peptide of the present invention. The binding of a peptide with another component (or second peptide) is well known in the art, and the binding may be by any known means in the conjugate of the present invention.

For example, when the binding is via a linker, known crosslinkers such as NHS ester, imide ester, maleimide, carbodiimide, allyl azide, diazirine, isocyanide, psoralen, etc. can be used. Depending on the crosslinker to be used, the peptide of the present invention may be modified as appropriate. For example, a cysteine can be added in advance to the C-terminal of the peptide of the present invention for binding with a maleimide linker.

A fluorescently labeled substance is conjugated to the above protein or peptide. The fluorescently labeled substance may be any substance that is activated (changes its physical properties) by irradiation with radiation, electromagnetic waves, or sound waves, for example, and emits fluorescence. The radiation includes radiation in a narrow sense, i.e., particle radiation such as beta rays, neutron rays, proton beams, heavy-ion beams, meson beams, etc., and electromagnetic radiation such as gamma rays and X-rays. The electromagnetic waves include so-called light rays such as infrared rays, visible rays, and ultraviolet rays, and radio waves, and the sound waves also include ultrasonic waves.

In a mechanism of cell membrane destruction according to the present invention, a drug becomes hydrophobic due to removal of a hydrophilic group called a ligand, for example, of the fluorescently labeled substance, and a failure occurs in the cell membrane, unlike the conventionally known photodynamic therapy (PDT). That is, in the present invention, the physical properties of the fluorescently labeled substance change in a state in which a fluorescently labeled substance-peptide conjugate binds to the marker molecule (on the cell membrane) that exists on a surface of the cell to be removed, and membrane-conjugate deformation or aggregate formation damage a cancer cell membrane.

In the present invention, a change of the physical properties of the fluorescently labeled substance becomes a “death switch”, and this switch can be turned on by a remote control of light that is not toxic to a living body, such as near-infrared light. It is a completely new cell killing method that can turn only the conjugate binding to a cancer cell into poison by light.

In the present invention, any substance that has the aforementioned “death switch” property and emits fluorescence can be used. However, a preferable fluorescently labeled substance is a photosensitive compound.

Phthalocyanine dye can be mentioned as a more preferable fluorescently labeled substance used in the present invention. Phthalocyanines are a group of photosensitizer compounds having a phthalocyanine ring system. Phthalocyanines are azaporphyrins that contain four benzoindole groups connected by nitrogen bridges in a 16-membered ring of alternating carbon and nitrogen atoms (i.e., C32H16N8) which form stable chelates with metal and non-metal cations. In these compounds, the ring center is occupied by a metal ion (either a diamagnetic or a paramagnetic ion) that may, depending on the ion, carry one or two ligands. In addition, the ring periphery may be either unsubstituted or substituted.

Phthalocyanines strongly absorb red or near infrared rays with absorption peaks falling between about 600 nm and 810 nm, which, in some cases, allow deep penetration of tissue by the light. Phthalocyanines are generally photostable. This photo stability is typically advantageous in pigments and dyes and in many of the other applications of phthalocyanines. The phthalocyanine dye has a maximum light absorption in the near infrared (NIR range). In some embodiments, the phthalocyanine dye has a maximum light absorption wavelength between 400 nm and 900 nm, such as between 600 nm and 850 nm or between 680 nm and 850 nm, for example at approximately 690±50 nm or 690±20 nm. In some embodiments, the phthalocyanine dye can be excited efficiently by commercially available laser diodes that emit light at these wavelengths.

In some embodiments, the phthalocyanine dye containing a reactive group is IR700 NHS ester, such as IRDye 700DX NHS ester (LiCor 929-70010, 929-70011).

Means for changing the physical properties of the fluorescently labeled substance can be irradiation with radiation, electromagnetic waves, or sound waves, for example, but is not limited thereto. It can also be done by chemical means. In the case of irradiation, irradiation with therapeutic doses of radiation or electromagnetic waves at a wavelength in the range of 400 nm to about 900 nm or about 400 nm to about 900 nm, 500 nm to about 900 nm or about 500 nm to about 900 nm, 600 nm to about 850 nm or about 600 nm to about 850 nm, 600 nm to about 740 nm or about 600 nm to about 740 nm, about 660 nm to about 740 nm, about 660 nm to about 710 nm, about 660 nm to about 700 nm, about 670 nm to about 690 nm, about 680 nm to about 740 nm, or about 690 nm to about 710 nm, for example, is performed. In some embodiments, cells, such as tumors, are irradiated with therapeutic doses of radiation or electromagnetic waves at a wavelength from 600 nm to 850 nm, such as 660 nm to 740 nm. In some embodiments, cells, such as tumors, are irradiated at a wavelength of at least 600 nm, 620 nm, 640 nm, 660 nm, 680 nm, 700 nm, 720 nm, or 740 nm, or at least about 600 nm, about 620 nm, about 640 nm, about 660 nm, about 680 nm, about 700 nm, about 720 nm, or about 740 nm, such as 690±50 nm or about 680 nm, for example.

In some embodiments, cells, such as tumors, are irradiated with a dose of at least 1 J/cm², such as at least 10 J/cm², at least 30 J/cm², at least 50 J/cm², at least 100 J/cm², or at least 500 J/cm². In some embodiments, the dose of irradiation is 1 to about 1000 or about 1 to about 1000 J/cm², about 1 to about 500 J/cm², about 5 to about 200 J/cm², about 10 to about 100 J/cm², or about 10 to about 50 J/cm². In some embodiments, cells, such as tumors, are irradiated with a dose of at least 2 J/m², 5 J/cm², 10 J/cm², 25 J/cm², 50 J/cm², 75 J/cm², 100 J/cm², 150 J/cm², 200 J/cm², 300 J/cm², 400 J/cm², or 500 J/cm², or at least about 2 J/cm², 5 J/cm², 10 J/cm², 25 J/cm², 50 J/cm², 75 J/cm², 100 J/cm², 150 J/cm², 200 J/cm², 300 J/cm², 400 J/cm², or 500 J/cm².

In some embodiments, cells, such as tumors, are irradiated or illuminated with a dose of at least 1 J/fiber length cm, such as at least 10 J/fiber length cm, at least 50 J/fiber length cm, at least 100 J/fiber length cm, at least 250 J/fiber length cm, or at least 500 J/fiber length cm. In some embodiments, the dose of irradiation is 1 to about 1000 or about 1 to about 1000 J/fiber length cm, about 1 to about 500 J/fiber length cm, about 2 to about 500 J/fiber length cm, about 50 to about 300 J/fiber length cm, about 10 to about 100 J/fiber length cm, or about 10 to about 50 J/fiber length cm. In some embodiments, cells, such as tumors, are irradiated with a dose of at least 2 J/fiber length cm, 5 J/fiber length cm, 10 J/fiber length cm, 25 J/fiber length cm, 50 J/fiber length cm, 75 J/fiber length cm, 100 J/fiber length cm, 150 J/fiber length cm, 200 J/fiber length cm, 250 J/fiber length cm, 300 J/fiber length cm, 400 J/fiber length cm or 500 J/fiber length cm, or at least about 2 J/fiber length cm, 5 J/fiber length cm, 10 J/fiber length cm, 25 J/fiber length cm, 50 J/fiber length cm, 75 J/fiber length cm, 100 J/fiber length cm, 150 J/fiber length cm, 200 J/fiber length cm, 250 J/fiber length cm, 300 J/fiber length cm, 400 J/fiber length cm or 500 J/fiber length cm.

In some embodiments, the dose of irradiation or illumination in a human subject is 1 to about 400 J/cm² or about 1 to about 400 J/cm², about 2 to about 400 J/cm², about 1 to about 300 J/cm², about 10 to about 100 J/cm², or about 10 to about 50 J/cm², e.g., at least 10 J/cm² or at least about 10 J/cm², 10 J/cm², within 10 J/cm² or within about 10 J/cm², 10 J/cm² or about 10 J/cm², at least 30 J/cm², at least 50 J/cm², or at least 100 J/cm². In some embodiments, the dose of irradiation in a human subject is 1 to 300 J/fiber length cm or about 1 to 300 J/fiber length cm, 10 to 100 J/fiber length cm or about 10 to 100 J/fiber length cm, or 10 to 50 J/fiber length cm or about 10 to 50 J/fiber length cm, e.g., at least 10 J/fiber length cm or at least about 10 J/fiber length cm, 10 J/fiber length cm, within 10 J/fiber length cm or within about 10 J/fiber length cm, 10 J/fiber length cm or about 10 J/fiber length cm, at least 30 J/fiber length cm, at least 50 J/fiber length cm, or at least 100 J/fiber length cm. In some cases, the dose of irradiation in human subjects for achieving PIT is less than the dose required for PIT in mice.

### Effect of the Invention

According to the present invention, among the cultured or isolated cells, the cell to be removed can be selectively removed (killed) non-invasively without damaging the object cell by using the antibody-photosensitizer conjugate and radiation, and thus it is possible to efficiently collect only the object cell.

Furthermore, it is possible to prevent omission of removal of the cell to be removed and omission of washing of the antibody-photosensitizer conjugate in the cell suspension, and thus the object cell can be normally cultured even after cell purification. In addition, the risk of tumorigenesis of cells transplanted into the patient can be greatly reduced, and thus contribution to the development of regenerative medicine and the development of effective treatment methods for diseases can be expected.

### BRIEF DESCRIPTION OF THE DRAWINGS

FIG. 1 A diagram of an embodiment of a flow-through purification device of the present invention.

FIG. 2 A flowchart showing the control contents of a control/processing unit.

FIG. 3 A diagram of an embodiment of a batch purification device of the present invention.

FIG. 4 A diagram showing a mechanism of cancer cell death.

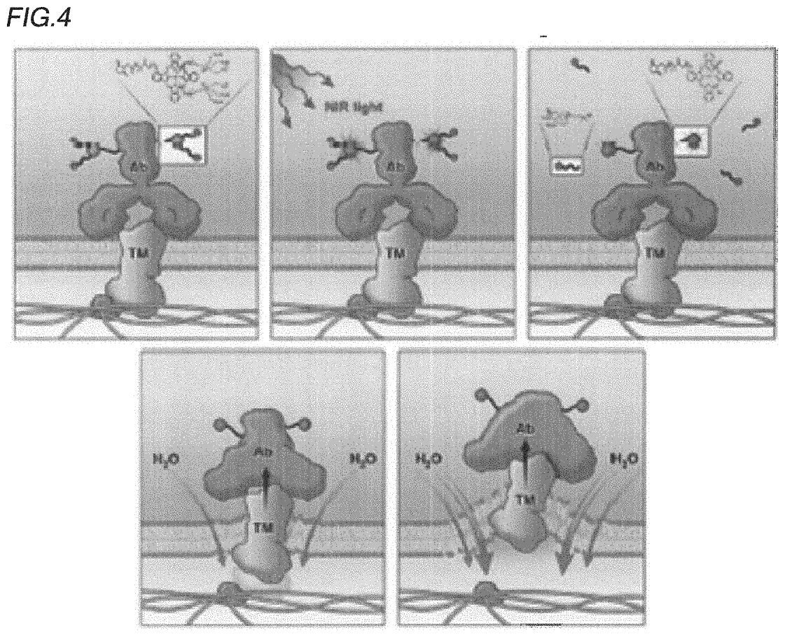

### MODES FOR CARRYING OUT THE INVENTION

### Embodiment 1: Flow-Through

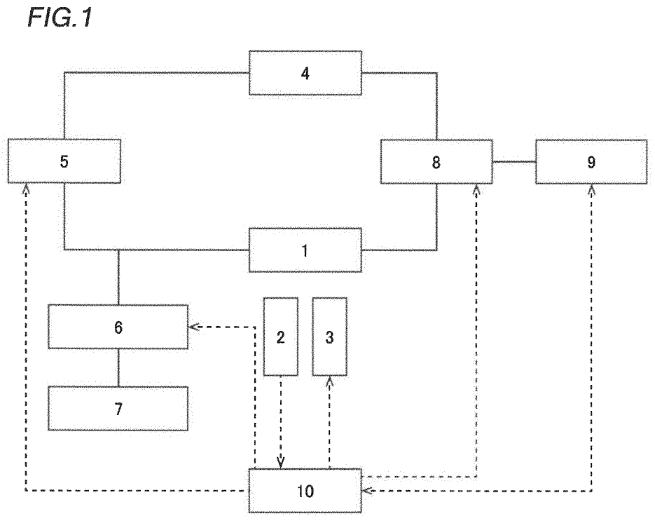

A flow-through purification device and a cell purification method using the device, which are an embodiment of the present invention, are described with reference to the accompanying drawings. FIG. 1 is a block diagram showing the configuration of a purification device of this embodiment.

As shown in FIG. 1, the purification device includes 1: holder, 2: detector, 3: radiation light source, 4: cell incubator, 5: liquid feeding pump, 6: introduction pump, 7: conjugate storage, 8: switching valve, 9: analyzer, and 10: control/processing unit. The control/processing unit 10 controls the energy intensity of radiation by the radiation light source 3, the amount of cell suspension fed by the liquid feeding pump 6, adjustment of the switching valve 8, analysis execution in the analyzer 9, etc. The fluorescence intensity detected by the detector 2 and analysis data obtained by the analyzer 9 are input to the control/processing unit 10. In general, at least some of the functions of the control/processing unit 10 can be performed by using a personal computer as a hardware resource and running dedicated control/processing software pre-installed on the personal computer on the computer.

The cell incubator 4 and the holder 1 are connected to each other by flow paths via the liquid feeding pump 6 and the switching valve 8. Furthermore, the analyzer 9 is connected to the holder 1 by a flow path by switching the switching valve 8. The cell incubator 4 may be detachable from these flow paths.

The analyzer 9 may analyze a cell suspension or only a culture medium of the cell suspension. When only the culture medium is analyzed, a filter (not shown) for collecting cells from the cell suspension is provided at any position in the flow path that connects the holder 1 to the switching valve 8 when the switching valve 8 is connected to the analyzer 9. The filter is manually or automatically arranged. Isopore Membrane Filter manufactured by Milipore (HTTP pore diameter: 0.4 μm, diameter: 47 mm, 047 00) can be used, for example, but the filter is not limited to this. The cells collected by the filter may be manually seeded in the cell incubator 4, or after the switching valve 8 is connected to the cell incubator 4, the cells may be automatically fed to the cell incubator 4 together with the cell suspension.

When the cells are subcultured in the cell incubator 4, they may be subcultured manually or automatically. As the culture medium used to culture the cells in the cell incubator 4, a culture medium commonly used to culture stem cells, e.g. DMEM/F12 or a culture medium containing DMEM/F12 as a main component (such as mTeSR1 or TeSR-E8), can be used when a target to be cultured is stem cells such as ES cells or iPS cells, but the culture medium is not limited to this.

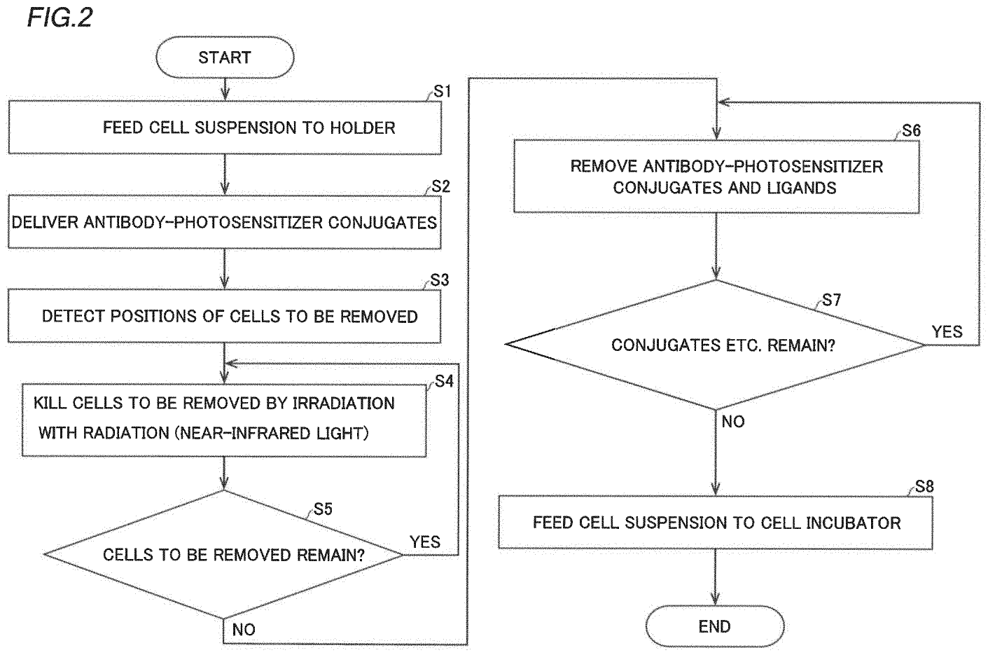

A purification procedure for purifying the cells using the purification device described above and a control by the control/processing unit 10 to perform the purification are now described based on a flowchart shown in FIG. 2.

A cell suspension contained in the cell incubator 4 and containing a culture medium and cells is fed to the holder 1 by the liquid feeding pump 6 (step 1). Upon completion of feeding of the cell suspension to the holder 1 or during the feeding, antibody-photosensitizer conjugates are delivered from the conjugate storage 7 by the introduction pump 6 (step 2). Then, the holder 1 is irradiated with radiation having enough energy to excite fluorescently labeled substances in the antibody-photosensitizer conjugates but not kill cells to be removed, and the positions of the cells to be removed in the holder are detected (step 3). The energy intensity of the radiation may be controlled by adjusting the irradiation time or irradiation diameter, for example, by the control/processing unit 10. Regarding the existence of the cells to be removed, for example, the cells to be removed may be detected by introducing the antibody-photosensitizer conjugates into cells known to be normal cells, storing the fluorescence intensity detected when radiation is radiated, comparing it with the fluorescence intensity detected in step 3, and making a determination based on whether or not the cells are to be removed.

Regarding the locations of the cells to be removed, positions assumed to be the cells to be removed may be detected on image data by capturing an observation image of the holder 1 in an imaging unit (not shown) such as a CCD camera via a reflecting mirror (not shown) and a condenser lens (not shown), inputting image data corresponding to the observation image reproduced in the imaging unit to the control/processing unit 10, introducing the antibody-photosensitizer conjugates into cells known to be normal cells, and comparing the fluorescence intensity detected when radiation is radiated with the fluorescence intensity detected in step 3. The control/processing unit 10 may control the radiation light source 4 to irradiate the detected locations of the cells to be removed with radiation. At that time, the radiation light source 4 may be moved to the irradiation position by a drive (not shown).

Furthermore, the image data may be displayed by a display (not shown).

Then, the positions of the cells to be removed detected in step 4 are irradiated with radiation having enough energy to kill the cells to be removed (step 4). Then, in order to detect whether or not the cells to be removed remain in the holder 1, radiation having enough energy to excite the fluorescently labeled substances in the antibody-photosensitizer conjugates but not kill the cells to be removed is radiated (step 5). When it is confirmed that the cells to be removed remain, the process operation in step 4 is performed again. When it is determined in step 5 that the cells to be removed have died in the holder 1, the antibody-photosensitizer conjugates and the ligands of the antibody-photosensitizer conjugates contained in the cell suspension are removed (step 6). Examples of a method for removing the antibody-photosensitizer conjugates and the ligands of the antibody-photosensitizer conjugates include a method for pellet collection or collection by a filter. After the cells to be removed have died, an object cell that remains in the cell suspension may be further washed with a serum-free medium, phosphate buffered saline, culture medium, or blocking buffer. Examples of the serum-free medium include EMEM and RPMI. Next, in order to determine whether or not the antibody-photosensitizer conjugates and the ligands of the antibody-photosensitizer conjugates remain in the cell suspension, the switching valve 8 is controlled such that the culture medium is fed to the analyzer 9, and the analyzer conducts an analysis (step 7). The analyzer 9 may include a liquid chromatograph mass spectrometer, and an ELISA method may be used. When it is determined based on the analysis result that the antibody-photosensitizer conjugates or the ligands of the antibody-photosensitizer conjugates remain, the process operation in step 6 is performed again. The “remain” refers to the fact that the antibody-photosensitizer conjugates or ligands of the antibody-photosensitizer conjugates in the cell suspension, for example, have a concentration that causes abnormal differentiation of cultured cells (e.g. a concentration in the cell suspension of 10% or more). The abnormalities here include tumorigenesis (teratoma, carcinogenesis), abnormal differentiation, chromosomal abnormalities, cell death, etc.

When it is determined from the analysis result obtained in step 7 that the antibody-photosensitizer conjugates and the ligands of the antibody-photosensitizer conjugates do not remain in the cell suspension, the switching valve 8 and the liquid feeding pump 5 are controlled such that the cell suspension is fed to the cell incubator 4 (step 8).

### Embodiment 2: Batch

The present invention is applicable not only to the flow-through purification device described above but also to a so-called batch purification device.

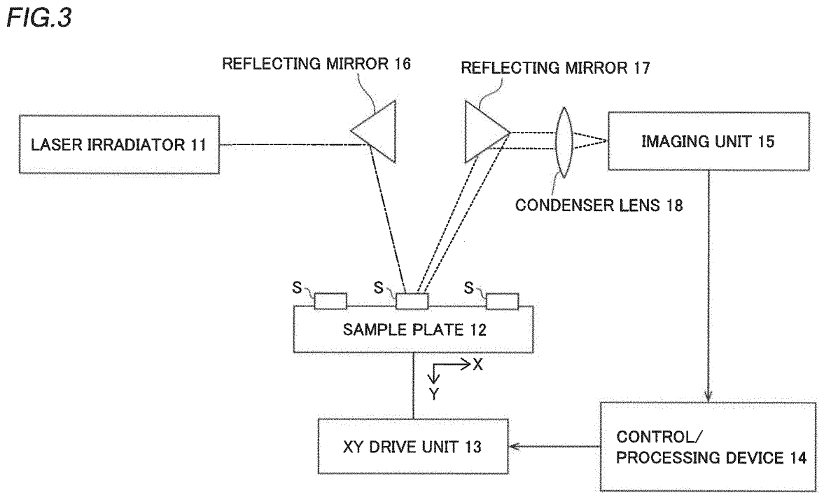

An example of a batch purification device is shown in FIG. 3.

This embodiment includes a sample plate 12 on which a plurality of cell culture vessels S are placed, a laser irradiator 11 that emits a laser beam, and a reflecting mirror 16 that reflects the laser beam and concentrates it on the cell culture vessels S placed on the sample plate 12. Observation images of the cell culture vessels S are taken into an imaging unit 15 such as a CCD camera via a reflecting mirror 17 and a condenser lens 18, and image data corresponding to the observation images reproduced by the imaging unit 15 is input to a control/processing unit 20.

The cell culture vessels S each contain a cell suspension containing a culture medium and cells, and antibody-photosensitizer conjugates. As an antibody photosensitizer, an antibody that selectively binds to a cell to be killed, such as a conjugate (Tra-IR700) of Tra (trastuzumab) and IR700 can be used.

The cell culture vessels S and the sample plate 12 are preferably placed in a CO₂ incubator (not shown). The CO₂ incubator is a well-known one capable of adjusting the CO₂ concentration and temperature of the atmosphere inside the incubator, and plays a role in maintaining the cell culture environment during the laser irradiation treatment, such as the pH of the culture medium filled in each of the cell culture vessels S, in a suitable state.

The control/processing unit 20 moves the sample plate 2 via an XY drive unit 13 such that each cell culture vessel S comes to a position at which the imaging unit 15 can capture an image. When the cell culture vessel S comes to the position at which it can be imaged, the imaging unit 15 acquires an image of the entire vessel and sends the image data to the control/processing unit 20. The control/processing unit 20 obtains a brightness value for each pixel of the acquired image. Then, using the obtained brightness values of all the pixels, a brightness value histogram showing the relationship between the brightness values and the frequency of occurrence of the pixels is created, and whether or not it is a cell region and whether or not it is a cell to which the antibody photosensitizer has bound are identified.

After the cell region is identified, a laser beam is radiated from the laser irradiator 11 toward the cell culture vessels S such that the antibody-photosensitizer conjugates that have bound to the cells to be killed in the cell culture vessels S act as death switches, and only specific cells that have bound to the antibodies can be killed.

The control/processing unit determines the presence or absence of dead cells based on the cell image captured by the imaging unit 15, and continues to radiate the laser beam from the laser irradiator when the cells to be removed are not killed.

### Other Embodiments

The present invention is not limited to the above embodiments, but an irradiated layer containing antibody-photosensitizer conjugates in a layer containing a material that receives and absorbs a laser beam may be provided on a cell culture vessel main body, such as a lid or a side surface of the vessel, for example. According to this, among cell colonies on the cell culture vessel, only unnecessary cells and cancer cells that have not differentiated into desired cells can be killed in a pinpointed manner, and a certain region in the irradiated layer of the cell culture vessel is raster-scanned with a laser beam such that all cells located in the region can be killed.

### DESCRIPTION OF REFERENCE NUMERALS

1: holder
2: detector
3: radiation light source
4: cell incubator
5: liquid feeding pump
6: introduction pump
7: conjugate storage
8: switching valve
9: analyzer
10: control/processing unit

1. A purification device configured to purify cells, the purification device comprising:
a holder configured to hold a cell suspension containing cells and an antibody-photosensitizer conjugate obtained by conjugating a targeting molecule that binds to a surface protein of a cell to be removed and a fluorescently labeled substance;
a radiation light source configured to radiate radiation to change a physical property of the fluorescently labeled substance contained in the holder;
a detector configured to detect abundance of the cells in the holder; and
a control/processing unit configured to control the radiant light source; wherein
the antibody-photosensitizer conjugate kills the cell to be removed by a change of the physical property of the fluorescently labeled substance; and
the control/processing unit is configured to determine whether or not the cell to be removed has been killed based on the abundance of the cells detected by the detector, and to continue irradiation with the radiation from the radiation light source when the cell to be removed has not been killed.

2. The purification device according to claim 1, wherein
a container containing the cell suspension containing the cells, a conjugate storage configured to store the antibody-photosensitizer conjugate, and the holder are connected by flow paths; and
the flow paths include pumps configured to introduce the cell suspension and the antibody-photosensitizer conjugate into the holder.

3. The purification device according to claim 1, wherein the cells include one or both of cultured cells and isolated cells.

4. The purification device according to claim 1, wherein the detector includes a fluorescence detector configured to detect fluorescence emitted from the fluorescently labeled substance, or a cell imager.

5. The purification device according to claim 1, wherein the control/processing unit is configured to radiate radiation having enough energy to excite the fluorescently labeled substance but not kill the cell to be removed before killing the cell to be removed by radiating radiation to change the physical property of the fluorescently labeled substance, and to detect a position of the cell to be removed in the holder and then irradiate the position with radiation having enough energy to kill the cell to be removed.

6. The purification device according to claim 1, wherein the control/processing unit is configured to determine whether or not the cell to be removed has died based on a presence of fluorescence detected by radiating radiation having enough energy to excite the fluorescently labeled substance but not kill the cell to be removed after radiating radiation having enough energy to kill the cell to be removed.

7. The purification device according to claim 1, wherein the holder includes a cell container containing the cell suspension containing the cells.

8. The purification device according to claim 1, further comprising:
a liquid feeding pump configured to flow the cell suspension; wherein
the holder forms a flow path including an inlet for taking in the cell suspension, an outlet for discharging the cell suspension containing purified cells, and the liquid feeding pump; and
the control/processing unit is configured to drive the liquid feeding pump to discharge the cell suspension in the flow path from the outlet when determining that removal of the cell to be removed has been completed based on the abundance of the cells detected by the detector.

9. The purification device according to claim 8, further comprising:
a cell container configured to house cells; wherein
the flow path forms a circulation flow path;
the cell container is provided in a return flow path of the circulation flow path; and
the control/processing unit is configured to drive the liquid feeding pump to feed the cell suspension in the flow path to the cell container when determining that the cell to be removed has died based on detected fluorescence abundance.

10. The purification device according to claim 9, comprising:
an analyzer configured to analyze the cell suspension containing the antibody-photosensitizer conjugate and a ligand of the antibody-photosensitizer conjugate;
a flow path configured to supply the cell suspension to the analyzer; and
a switching valve configured to selectively switch any flow path to the analyzer or the cell container; wherein
the switching valve is provided in the return flow path of the circulation flow path.

11. The purification device according to claim 10, wherein the analyzer includes a liquid chromatograph mass spectrometer.

12. The purification device according to claim 1, wherein the radiation radiated by the radiation light source has a wavelength of 660 to 740 nm.

13. The purification device according to claim 1, wherein
the radiation radiated by the radiation light source includes near-infrared light; and
the fluorescently labeled substance activated by irradiation with the near-infrared light includes a phthalocyanine dye.

14. The purification device according to claim 13, wherein the phthalocyanine dye is IRDye® 700.

15. The purification device according to claim 1, wherein a ligand of the antibody-photosensitizer conjugate is a C14H33NO10S3Si fragment.
